SEQUENCE LISTING (22 sequences)

SEQ ID NO: 1 - DNA, 14 bp, Artificial Sequence
  1TM
ctacgccacaagct

SEQ ID NO: 2 - DNA, 15 bp, Artificial Sequence
  1Tw
cctacgccaccagct

SEQ ID NO: 3 - DNA, 13 bp, Artificial Sequence
  2TM
acgccaacagctc

SEQ ID NO: 4 - DNA, 15 bp, Artificial Sequence
  2Tw
ctacgccaccagctc

SEQ ID NO: 5 - DNA, 14 bp, Artificial Sequence
  2CM
ctacgccagcagct

SEQ ID NO: 6 - DNA, 14 bp, Artificial Sequence
  N2C
cctacgccagcagc

SEQ ID NO: 7 - DNA, 15 bp, Artificial Sequence
  5AM
ctggtgacgtaggca

SEQ ID NO: 8 - DNA, 14 bp, Artificial Sequence
  5Aw
tggtggcgtaggca

SEQ ID NO: 9 - DNA, 14 bp, Artificial Sequence
  N1A
ttggagctagtggc

SEQ ID NO: 10 - DNA, 14 bp, Artificial Sequence
  N1Aw
ttggagctggtggc

SEQ ID NO: 11 - DNA, 14 bp, Artificial Sequence
  N2A
ctgatggcgtaggc

SEQ ID NO: 12 - DNA, 14 bp, Artificial Sequence
  N1C
ttggagctcgtggc

SEQ ID NO: 13 - DNA, 16 bp, Artificial Sequence
  1Cw
ttggagctggtggcgt

SEQ ID NO: 14 - DNA, 27 bp, Artificial Sequence
  Forward primer 1F
tgactgaatataaacttgtggtagttg

SEQ ID NO: 15 - DNA, 22 bp, Artificial Sequence
  Reverse Primer AS12R
gctgtatcgtcaaggcactctt

SEQ ID NO: 16 - DNA, 66 bp, Artificial Sequence
  wild-type amplicon
tgactgaatataaacttgtggtagttggagctggtggcgtaggcaagagtgccttgacgatacagc

SEQ ID NO: 17 - DNA, 124 bp, Homo sapiens
aggcctgctgaaaatgactgaatataaacttgtggtagttggagctggtggcgtaggcaagagtgccttgacgatacagctaattcagaatcattttgtggacgaatatgatccaacaatagag

SEQ ID NO: 18 - DNA, 15 bp, Artificial Sequence
  1AM
ctacgccactagctc

SEQ ID NO: 19 - DNA, 16 bp, Artificial Sequence
  1CM
ttggagctcgtggcgt

SEQ ID NO: 20 - DNA, 15 bp, Artificial Sequence
  2AM
tggagctgatggcgt

SEQ ID NO: 21 - DNA, 25 bp, Artificial Sequence
  AS12F
aggcctgctgaaaatgactgaatat

SEQ ID NO: 22 - DNA, 22 bp, Artificial Sequence
  1R
tcgtccacaaaatgattctgaa

(19) United States
(12) Patent Application Publication
(10) Pub. No.: US 2013/0217020 A1
(43) Pub. Date: Aug. 22, 2013

(54) METHODS FOR IMPROVING SENSITIVITY AND SPECIFICITY OF SCREENING ASSAYS OF KRAS CODONS 12 AND 13 MUTATIONS

(71) Applicants: Li-Hui Chow, Miaoli (TW); Tsang-Wu Liu, Miaoli County (TW)

(72) Inventors: Li-Hui Chow, Miaoli (TW); Tsang-Wu Liu, Miaoli County (TW)

(73) Assignee: NATIONAL HEALTH RESEARCH INSTITUTES, Miaoli County (TW)

(21) Appl. No.: 13/402,779

(22) Filed: Feb. 22, 2012

(51) Int. Cl. C12Q 1/68

(52) U.S. Cl. 435/6.12

(57) ABSTRACT

A method of diagnosing a KRAS gene mutation at codons 12-13 in a DNA sample is disclosed. The method comprises detecting one or more than one mutation in the KRAS gene codons 12-13 of the DNA sample by performing an allelic discrimination assay using a mutant probe, a wild-type probe paired with the mutant probe, a forward primer and a reverse primer, the mutant probe being adapted to detect a single nucleotide mutation at 1A, 1T, 1C, 2A, 2T, 2C or 5A of the KRAS gene codons 12-13 of the DNA sample, and the primers each having no greater than 25 nucleotides in length are adapted to amplify a region spanning KRAS exon 2 codons 12-13, wherein the mutant and wild-type probes are labeled with different fluorescent dyes.

### FIELD OF THE INVENTION

The invention relates to assays for detecting KRAS c12-13 mutational genotypes which are diagnostic and/or prognostic of cancer with high specificity and sensitivity.

### BACKGROUND OF THE INVENTION

KRAS (official gene name: v-Ki-ras2 Kirsten rat sarcoma viral oncogene homolog; aliases: KRAS2, RASK2; Gene Bank Accession Number NM_{—}033360, which is incorporated herein by reference in its entirety) is a small GTPase activated by EGFR signaling that plays an important role in the intracellular signaling pathways of proliferation, survival, and differentiation. As EGFR being considered to be involved in the pathogenesis of most epithelial cancers, anti-EGFR drugs are anticipated to improve outcomes of millions of patients worldwide K-ras mutations transform the intrinsic GTPase activity of the protein causing the constitutively active, GTP-bound conformation. This permanently activated (mutated) K-ras protein downstream of EGFR may counteract therapeutic targeting of the EGFR.

The missense single nucleotide substitutions of K-ras gene, predominantly at codons 12 and 13 (>98%), often occur in common epithelial malignancies such as pancreas cancer (75-90%), lung adenocarcinomas (20-50%), and colorectal cancer (CRC, 30-60%). Patients with K-ras mutations are unlikely to benefit from anti-EGFR therapies, such as panitumumab and cetuximab (VECTIBIX® and ERBITUX®). KRAS testing has been recommended to guide therapy in patients with lung adenocarcinoma as well as CRC. Assessment of KRAS mutation status may also provide predictive values to cancer progression and aggression, as well as hereditary predisposition to CRC. In addition, detection of KRAS mutations in plasma DNA can be a useful tool to evaluate tumor burden and efficacy of treatment for patients with pancreas cancer. Analysis of tumor DNA in bile, such as K-ras c12-13 mutations, may be an ancillary testing for diagnosis of early biliary tract carcinoma.

While screening methods for rapid identification of KRAS hot-spot mutations are undergoing dynamic development, little is known as to which combination of probe and primer sequences discriminate the allele best and contribute to specificity.

### SUMMARY OF THE INVENTION

In one aspect, the invention relates to a method of diagnosing a KRAS gene mutation at codons 12-13 in a DNA sample, which comprises detecting one or more than one mutation in the KRAS gene codons 12-13 of the DNA sample by performing an allelic discrimination assay using a mutant probe, a wild-type probe paired with the mutant probe, a forward primer and a reverse primer. The mutant probe is adapted to detect single nucleotide mutations at 1A, 1T, 1C, 2A, 2T, 2C or 5A of the KRAS gene codons 12-13 of the DNA sample, and the primers each having no greater than 25 nucleotides in length are adapted to amplify a region spanning KRAS exon 2 codons 12-13, wherein the mutant and wild-type probes are labeled with different fluorescent dyes.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1 shows monthly quality control (QC) of the K-ras allele discrimination assay, including wild-type (wt) and 5% mutant (mt) plasmid DNA, which were monitored during the services to CRC patients (Chow et al).

FIG. 2 shows the sensitivity of K-ras assay improved from detecting 5% 2A mutant (mt) in wild-type (wt) plasmid (the lowest distinguishable percentage among those indicated in A) to 1% (B) not only by new 2A probes, N2A (CTGATGGCGTAGGC; SEQ ID NO: 11) mt probe paired with TTGGAGCTGGTGGC (SEQ ID NO: 10) wt probe, but also by the primer 1F (TGACTGAATATAAACTTGTGGTAGTTG; SEQ ID NO: 14) paired with AS12R (GCTGTATCGTCAAGGCACTCTT; SEQ ID NO: 15). Neither did the new probes with primer pairs of 1F/1R (TCgTCCACAAAATgATTCTgAA (SEQ ID NO: 22) or AS12F (AGGCCTGCTGAAAATGACTGAATAT (SEQ ID NO: 21)/AS12R separate the 2A allele as well.

FIG. 3 shows in spite of a similar trend as N2A probe in primary test (A), N2C mt probe (CC TACGCCAGCAGC; SEQ ID NO: 6) with the wt probe (CCTACGCCACCAGCT; SEQ ID NO: 2) and primers 1F (TGACTGAATATAAACTTGTGGTAGTTG; SEQ ID NO: 14) and AS12R (GCTGTATCGTCAAGGCACTCTT; SEQ ID NO: 15) separated 5% 2C and 2C5A mutant (mt) plasmid DNA, and the genomic DNA (gDNA) of 2C containing RPMI-8226 cell from wild-type (wt) no better than old 2C probe (CTACGCCAGCAGCT; SEQ ID NO: 5) as measured by the Y differences (ΔY) (B). The primer pairs AS12F (AGGCCTGCTGAAAATGACTGAATAT; SEQ ID NO: 21) and AS12R separated 2C allele much better than 1F (SEQ ID NO: 14) and 1R primers (TCgTCCACAAAATgATTCTgAA; SEQ ID NO: 22) with either new or old probes (A).

FIG. 4A shows except 2C the full scale K-ras c12-13 assay using the new probe and primer sets detecting 5% mutants as the least sensitivity; and as an example, N1C (TTGGAGCTCGTGGC; SEQ ID NO: 12) mt probe and TTGGAGCTGGTGGCGT (SEQ ID NO: 13) wt probe discriminated the allele better (monitored in 2009 June and July) (B).

FIG. 5 shows sensitivity of the K-ras c12-13 assay using new probes and primers set with the TaqMan Fast Universal PCR Master Mix of ABI reagent (solid bars) detecting 1 and 2% mutants better and also achieveable by a cheaper reagent called KAPA Probe Fast qPCR Kit (empty bars) (A). KAPA's comparable Y differences (ΔY) between mutants (MT) and wild-type (WT) with new 1A, 1C, and 2A probes (in circles) were repeated in separate experiments with only 1% mutants plasmid DNA (B).

FIG. 6 shows sensitivity of the K-ras assay using new primers and probes set compared between ABI 7900 (A) and 7500 (B) instrument. Both detected 1% and 2% mixtures of K-ms c12-13 mutants (1A, 1T, 1C, 2A, 2T, 2C, and 5A on the X axis).

FIG. 7 shows sensitivity of the K-ras assay using new primers and probes set compared between 10 and 20 ng of cell's gDNA used per allele reaction. Results shows 10 ng is comparable to 20 ng. 1C result is omitted because no cells were found with 1C mutant. For 1A mutant detection, probes N1A and N1Aw were used. Mutant 2A were detected with probes N2A and N2Aw. Mutants 1T, 2T, 2C and 5A were detected with probes 1TM and 1Tw; 2TM and 2Tw; 2CM and 2Cw; and 5AM and 5Aw as shown in Table 1, respectively.

FIG. 8 shows the K-ras assay of new primers and probes set detecting alleles in two co-cultured cells, for example, 5A of Hone1 co-cultured with DLD1 (H+D), corresponding to the allele of DLD1 cell, with 10 ng gDNA. Mutants 2T, 1A and 5A were detected with probes N1A and N1Aw; 2TM and 2Tw; and 5AM and 5Aw as shown in Table 1, respectively.

FIG. 9 shows the K-ras assay of new primers and probes set discriminating the alleles in two co-cultured cells, for example, 2T and 5A in SW480 co-cultured with DLD1 (S+D), corresponding to respective alleles of SW480 and DLD1 cells, with 100 ng cDNA. However, the signal of A549's 1A was weakened during its co-culturing with Hone1 or DLD1 but not SW480. Possibility of gene expression being interfered differently during co-culturing cannot be ruled out.

FIG. 10 shows no mutant detected in serum DNA (20 ng) of 6 CRC patients vs. 2T and 5A mutants detected in the gDNA of patient 120's and 1305's tumor (T; non-tumor, N) among 5 available patients; and a 2A2T double mutant detected in the cDNA (100 ng) of patient 178's frozen tissue (T and N) but lack of the gDNA. No mutants were detected in serum DNA of 178 and of 5 other patients.

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, when a number or a range is recited, ordinary skill in the art understand it intends to encompass an appropriate, reasonable range for the particular field related to the invention.

As used herein, “a region spanning KRAS exon 2 codons 12-13” means “a nucleotide sequence that comprises the sequence of KRAS gene exon 2 condons 12-13.”

As used herein, the term “paired probes” or “a pair of probes” shall mean a pair of probes consisting of a mutant probe and a wild-type probe, in which the wild-type probe is designed to pair with the mutant probe.

A QC mutant contains the same DNA sequence as a corresponding mutant probe and the amount of the QC mutant used may be about 1˜5% of the amount of the DNA sample used in parallel PCR. For example, when the mutant probe 2CM (SEQ ID NO: 5) is used, the QC mutant shall comprise or consist of the nucleotide sequence of SEQ ID NO: 5, and when the mutant probe N1A (SEQ ID NO: 9) is used, the QC mutant shall comprise or consist of the nucleotide sequence of SEQ ID NO: 9, and etc.

As used herein, the term “a comparable intensity” shall mean that the fluorescent signal intensity obtained from a DNA sample has no significant difference from the intensity obtained from a corresponding quality control (QC) mutant. A comparable intensity means the fluorescent intensity obtained from a DNA sample is the same as that from a QC mutant. The intensities for the DNA sample and the QC mutant may be measured by performing parallel PCRs on the DNA sample and the QC mutant.

Hybridization with allele specific oligonucleotide (ASO) utilizes the principle that a single-base mismatch results in a lower binding energy and melting point temperature (Tm). ASO may be deployed in a traditional format of dot blot, fluorescent labeled probes of real-time PCR (qPCR) as well as microarray DNA chips. The strength of binding motifs is determined by sequence-based Tm of the probe as well as unfolding of secondary structure of the amplicon. Therefore, both sequences of the probe and length or structure of the amplicon play an important role in the ASO based clinical assay.

To amplify and detect the individual K-ras c12-13 variants, the oligonucleotide sequences specific for allele discrimination including amplification primers for the gene region and hybridization probes for the mutations to identify are provided using a platform of real-time PCR (qPCR). In a preferred embodiment the shorter probes (13-20 mers) with Taqman minor-groove-binder (MGB) are utilized to achieve a greater melting temperature difference (ΔTm). With straightforward real-time PCR reaction, the instant invention optimizes probe and primer sequences surrounding the codons 12-13 area at K-ras exon 2 to improve performance of the K-ras c12-13 assay as a format of allele-specific oligonucleotides (ASO).

Conclusive K-ras genotyping with qPCR depends on the ability to discriminate different mutant alleles from wild-type. There are two challenges to achieving this goal: one is the heterogeneity of testing materials, the other is the detection limits of the discrimination assay. In addition to unequal molar amount wild-type and mutant DNA in template mixture that contains variable contents of tumor versus non-tumor area, a heterozygous or homozygous mutation increases the genetic heterogeneity of the tissue. Currently, the most appropriate material for K-ras mutation testing is primary tumor tissue, which is commonly archived, accessible, and contains sufficient amount of carcinoma cells required for testing. As to endoscopic biopsy of primary tumor, sufficient carcinoma cells must be identified in the area. In instance of patients with metastatic disease, 20% is estimated have no archived primary tumor, thereby, material from the metastatic tumor, such as the resected liver metastases or positive lymph node, can be used to perform the test. However, discordant K-ras status is not uncommon between primary and metastatic tumor tissues (Mariani et al. (2010) “Concordant analysis of KRAS status in primary colon carcinoma and matched metastasis. Anticancer Res” 30, 4229-35).

Optimization of allele-specific probes and primers cannot be based on estimation of melting temperature (Tm) alone, since affinities to the target sequences may differ between probes as well as amplicon's secondary structures, especially vicinity around the binding motifs. Primer Express software v1.3 (ABI) was used at first to search for the sequences of primers (e.g., AS12F and AS12R), and some probes, however, outcomes were weighed and improved through parallel testing and substituting the components individually, such as probes, primers, Taq DNA polymerase, and lastly the automatic qPCR instrument. See Chow, L. et al. (2012) “Differences in the frequencies of K-ras c12-13 genotypes by gender and pathologic phenotypes in colorectal tumors measured using the allele discrimination method. Environmental and Molecular Mutagenesis” 53: 22-31, which is incorporated herein by reference in its entirety. Furthermore, the amounts and the types of template were tested to confirm the optimized components.

Taq DNA polymerases includes, but not limited to, TaqMan Fast Universal PCR Master Mix (Applied Biosystems, CA, USA) and KAPA Probe Fast qPCR Kit (Kapa Biosystems, MA, USA).

The instruments of qPCR include, but not limited to, ABI 7900 and ABI 7500 Fast.

### Clinical Diagnostic Applications:

Human DNA includes genomic DNA (gDNA) and complementary DNA (cDNA) reversely transcribed from mRNA of fresh tissue. In one embodiment of the invention, gDNA of FFPE human tissue is extracted with a DNeasy Blood & Tissue Kit (Qiagene, Valencia, Calif., USA). Alternatively, cDNA reversely transcribed from the mRNA extract of frozen tissues or cancer cells with Trizol (Invitrogen, NY, USA) may be utilized. Simultaneous detections of 7 alleles are operated for each specimen, and the individual QC mutants must be included in each run for result interpretations regardless how many specimens (up to 11 as maximum in an 8×12-well plate) in the run. Reagents in all 7 reactions are the same except the allele specific probes.

As a perspective approach in clinical usage, the instant invention may be applied to serum DNA that is ample and freshly prepared by adequate extraction reagents known in the art.

In one aspect, the invention relates to a method of diagnosing a KRAS gene mutation at codons 12-13 in a DNA sample, which comprises detecting one or more than one mutation in the KRAS gene codons 12-13 of the DNA sample by performing an allelic discrimination assay using a mutant probe, a wild-type probe paired with the mutant probe, a forward primer and a reverse primer. The mutant probe is adapted to detect a single nucleotide mutation at 1A, 1T, 1C, 2A, 2T, 2C or 5A of the KRAS gene codons 12-13 of the DNA sample, and the primers each having no greater than 25 nucleotides in length are adapted to amplify a region spanning KRAS exon 2 codons 12-13, wherein the mutant and wild-type probes are labeled with different fluorescent dyes.

The mutant and wild-type probes are a pair selected from the group consisting of:
(i) SEQ ID NO: I paired with SEQ ID NO: 2;
(ii) SEQ ID NO: 3 paired with SEQ ID NO: 4;
(iii) SEQ ID NO: 5 paired with SEQ ID NO: 2;
(iv) SEQ ID NO: 7 paired with SEQ ID NO: 8;
(v) SEQ ID NO: 9 paired with SEQ ID NO: 8 or 10;
(vi) SEQ ID NO: 11 paired with SEQ ID NO: 10; and
(vii) SEQ ID NO: 12 paired with SEQ ID NO: 13.

In one embodiment of the invention, the detecting step comprises:
(a) admixing the DNA sample with the paired probes, the primers and polymerase chain reaction (PCR) reagents;
(b) amplifying the region spanning the codons 12-13 of the KRAS exon 2;
(c) measuring the intensity of the fluorescent dye of the mutant probe in the DNA sample; and
(d) comparing the intensity to that of a corresponding quality control (QC) mutant, wherein a comparable or an increased intensity indicates the presence of the mutation.

In another embodiment of the invention, the forward and reverse primers comprise the nucleotide sequences of SEQ ID NOs: 14 and 15, respectively.

In another embodiment of the invention, the aforementioned method comprises detecting mutations at 1A, 1T, 1C, 2A, 2T, 2C and 5A of the KRAS gene codons 12-13, wherein the paired probes comprises the following pairs:
(i) SEQ ID NO: 1 paired with SEQ ID NO: 2;
(ii) SEQ ID NO: 3 paired with SEQ ID NO: 4;
(iii) SEQ ID NO: 5 paired with SEQ ID NO: 2;
(iv) SEQ ID NO: 7 paired with SEQ ID NO: 8;
(v) SEQ ID NO: 9 paired with SEQ ID NO: 8 or 10;
(vi) SEQ ID NO: 11 paired with SEQ ID NO: 10; and
(vii) SEQ ID NO: 12 paired with SEQ ID NO: 13.

In another embodiment of the invention, the mutant probe is labeled with FAM.

In another embodiment of the invention, the wild-type probe is labeled with VIC.

In another embodiment of the invention, each probe has no greater than 18 or 16 nucleotides in length.

In another embodiment of the invention, the primers are adapted to amplify an amplicon of less than 80 or 70 base pairs.

In another embodiment of the invention, the primers are adapted to amplify a DNA fragment comprising the nucleotide sequence of SEQ. ID NO: 16.

In another embodiment of the invention, the primers are adapted to amplify a DNA fragment consisting of the nucleotide sequence of SEQ ID NO: 16.

In another embodiment of the invention, the amplifying step comprises performing a real time PCR.

In another embodiment of the invention, the DNA sample comprises a gDNA, or cDNA, prepared from a specimen of a tumor biopsy, a paraffin-embedded tumor tissue section (FFPE), a fresh or frozen tumor, or a tumor cell line.

In another embodiment of the invention, the tumor biopsy is obtained from a patient with colorectal cancer (CRC), mucinous or metastatic tumor, or cholangiocarcinoma.

In another embodiment of the invention, the cDNA is prepared from a frozen tumor tissue.

In another embodiment of the invention, the DNA sample comprises a serum DNA from a CRC patient.

In another embodiment of the invention, the amount of gDNA sample is no more than 10 ng/μl.

In another embodiment of the invention, the amount of DNA sample is no more than 0.1 ng/μl.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Specimens Collection and Allele Discrimination Analysis of 7 KRAS Mutations

Genomic DNA isolated from a total of 21 cancer cell lines (colorectal or lung cancer) were sequenced and tested. Tumor samples were collected from 204 CRC patients diagnosed between 2007 and 2009 with informed consent. All tumor sample specimens were stained with Hematoxylin and Eosin and re-evaluated by a pathologist at the National Institute of Cancer Research, National Health Research Institutes, Taiwan, R.O.C. This study was approved by the Human Experiment and Ethics Committee of National Cheng Kung University Hospital.

Fam-labeled mutant probes for 1A, 1T, 1C, 2A, 2T, 2C, and 5A, and Vic-labeled unidirectional wild-type probe were designed (TABLE 1), simultaneously detecting both mutant (Y) and wild-type (X) alleles. To each real-time PCR reaction of 1×TaqMan Genotyping Master Mix (P/N 4352042, Applied Biosystems) or 1×KAPA Probe Fast qPCR Kit Master Mix Universal (KK4701, Kapa Biosystems) the following were added: 20 or 10 ng of gDNA, or 100 ng of RNA-reverse transcribed cDNA, 3.38 pmole of paired Fain- and Vic-labeled probes, and 9 pmole of primers. An ABI PRISM 7900HT or 7500Fast Sequence Detection System (Applied Biosystems) was programmed as follows: stabilization at 50° C. for 2 min, 95° C. for 10 min, and 40 cycles of 95° C. for 15 sec and 60° C. for 60 sec. Mixtures of 1-5% mutant DNA plasmids in wild-type DNA plasmids, constructed by site directed mutagenesis, were used as positive controls. For each experiment, the no-template control reactions (NTC) were included as the negative control.

All fluorescent labeled probes used against individual mutant alleles of the K-ras gene are listed below.

TABLE 1
Paired Probes

| | Fam-1 labeled | | Vic-1 labeled |
|---|---|---|---|
| Name | mutant probe | Name | wild-type probe |
| 1AM | CTACGCCACTAGCTC | 2Tw | CTACGCCACCAGCTC |
| | (SEQ ID NO: 18) | | (SEQ ID NO: 4) |
| 1CM | TTGGAGCTCGTGGCGT | 1Cw | TTGGAGCTGGTGGCGT |
| | (SEQ ID NO: 19) | | (SEQ ID NO: 13) |
| 2AM | TGGAGCTGATGGCGT | 1Cw | TTGGAGCTGGTGGCGT |
| | (SEQ ID NO: 20) | | (SEQ ID NO: 13) |
| N2C | CCTACGCCAGCAGC | 1Tw | CCTACGCCACCAGCT |
| | (SEQ ID NO: 6) | | (SEQ ID NO: 2) |
| 1TM | CTACGCCACAAGCT | 1Tw | CCTACGCCACCAGCT |
| | (SEQ ID NO: 1) | | (SEQ ID NO: 2) |
| 2TM | ACGCCAACAGCTC | 2Tw | CTACGCCACCAGCTC |
| | (SEQ ID NO: 3) | | (SEQ ID NO: 4) |
| 2CM | CTACGCCAGCAGCT | 1Tw | CCTACGCCACCAGCT |
| | (SEQ ID NO: 5) | | (SEQ ID NO: 2) |
| 5AM | CTGGTGACGTAGGCA | 5Aw | TGGTGGCGTAGGCA |
| | (SEQ ID NO: 7) | | (SEQ ID NO: 8) |
| N1A | TTGGAGCTAGTGGC | N1Aw | TTGGAGCTGGTGGC |
| | (SEQ ID NO: 9) | | (SEQ ID NO: 10) |
| | | | or |
| | | | SAW |
| | | | (SEQ ID NO: 8) |
| N2A | CTGATGGCGTAGGC | N1Aw | TTGGAGCTGGTGGC |
| | (SEQ ID NO: 11) | | (SEQ ID NO: 10) |
| N1C | TTGGAGCTCGTGGC | 1Cw | TTGGAGCTGGTGGCGT |
| | (SEQ ID NO: 12) | | (SEQ ID NO: 13) |

Letter M in probe's name means mutant, while letter W and w mean wild-type; letter N means new probes made. The underlined indicate the mutant sequences detected by the probes; underlined sequences in bold mean in complementary to the detected mutants.
In Table 1, probes 1TM, 2TM, 2CM, 5AM detected mutants 1T, 2T, 2C and 5A respectively, and new probes N1A, N2A and A1C detected mutants 1A, 2A and 1C, respectively, with the detected single nucleotide mutation underlined.

PCR primers: Primer sequences tested were as follows:

Forward Primers: 1F:
(SEQ ID NO: 14)
TGACTGAATATAAACTTGTGGTAGTTG;
AS 12F:
(SEQ ID NO: 21, framed in the front
sequence of exon 2 below).
AGGCCTGCTGAAAATGACTGAATAT
Reverse Primer: AS12R:
(SEQ ID NO: 15)
GCTGTATCGTCAAGGCACTCTT;
1R:
(SEQ ID NO: 22; framed close to the
back sequence of exon 2 below)
TCgTCCACAAAATgATTCTgAA

The verified (underlined) good primers synthesize an amplicon of 66 bp, the sequence of wild-type amplicon is:

(SEQ ID NO: 16)
TGACTGAATATAAACTTGTGGTAGTTGGAGCTGGTGGCGTAGGCAAGAGTGCCTTGACGAT
ACAGC,
in which the underlined are localizations of 1F and AS12R
(complementary) sequences.

Alternatively, the possible mutant amplicon sequences that may be synthesized by 1F and AS12R are represented by the following sequence: tgactgaa tataaacttg tggtagttgg agctNNtgAc gtaggcaaga gtgccttgac gatacagc, in which the letters “NN” represent any nucleotide (ACT) at the position beside the wild-type's G.

The combination of primers 1F and AS12R with new probes for K-ras exon 2 improved the assay of 7 most common K-ras c12-13 mutant alleles.

K-Ras NM_{—}004985 exon2 sequence is as follows:

(SEQ ID NO: 17)

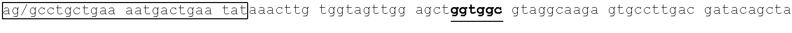

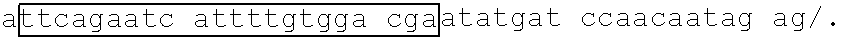

The codon 12 region is ggt, the codon 13 region is ggc. The framed sequences are locations of AS12F and 1R (complementary). The sequence of SEQ ID NO: 17 encompasses the sequence of SEQ ID NO: 16.

For each PCR reaction, final concentrations of individual components in a 15 μl total reaction volume are shown below.

TABLE 2

| | |
|---|---|
| *DNA | 10 to 20 ng gDNA, 100 ng cDNA, or serum DNA |
| Primers | 200-600 nM |
| Fam-probe | 200-400 nM |
| Vic-probe | 100-200 nM |
| dNTPs, MgCl2 and | Contained in 2X TaqMan Fast Universal PCR |
| Enzyme | Master Mix or KAPA Probe Fast qPCR Kit Master |
| | Mix Universal: 1X |

*For QC DNA, 1% or 5% mutant plasmid DNA in a background of wild-type was used. For non-template control (NTC), no template DNA was present in the reaction mixture. For each allele, a PCR reaction on the QC mutant DNA was performed in parallel to the sample DNA using the same allele-specific probes.

TABLE 3 catalogs the mutations that were assayed.

TABLE 3

| KRAS | Mutation |
|---|---|
| Codon 13 | Position 5, G to A |
| Codon 12 | Position 1, G to A |
| Codon 12 | Position 1, G to C |
| Codon 12 | Position 1, G to T |
| Codon 12 | Position 2, G to A |
| Codon 12 | Position 2, G to C |
| Codon 12 | Position 2, G to T |

### Data Analysis:

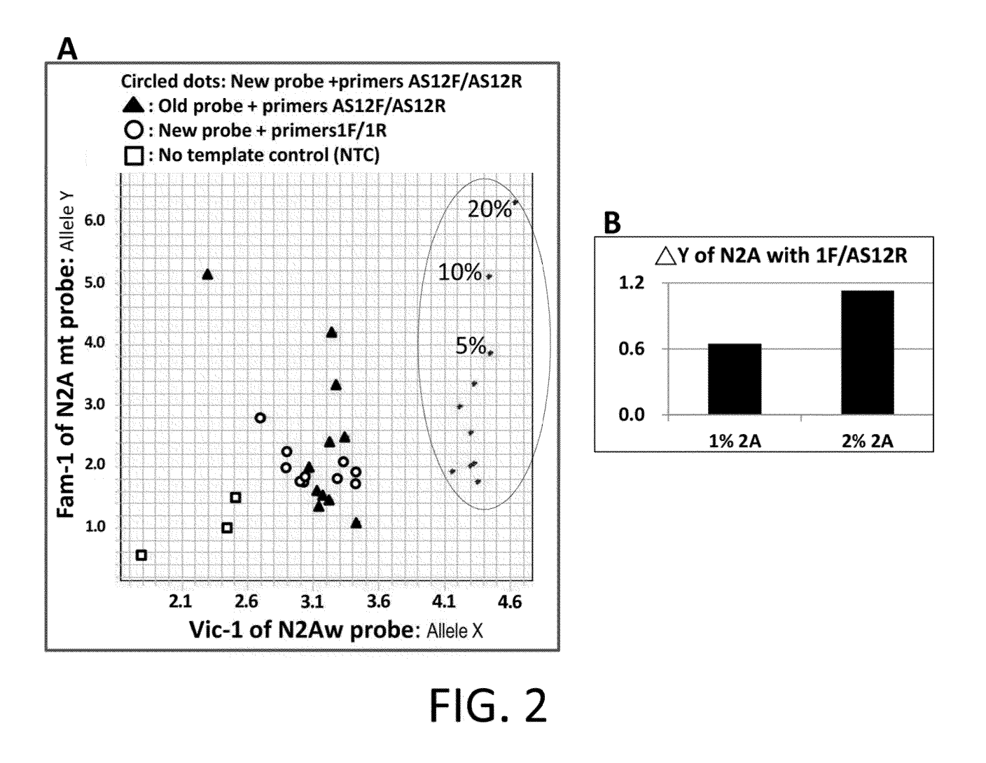

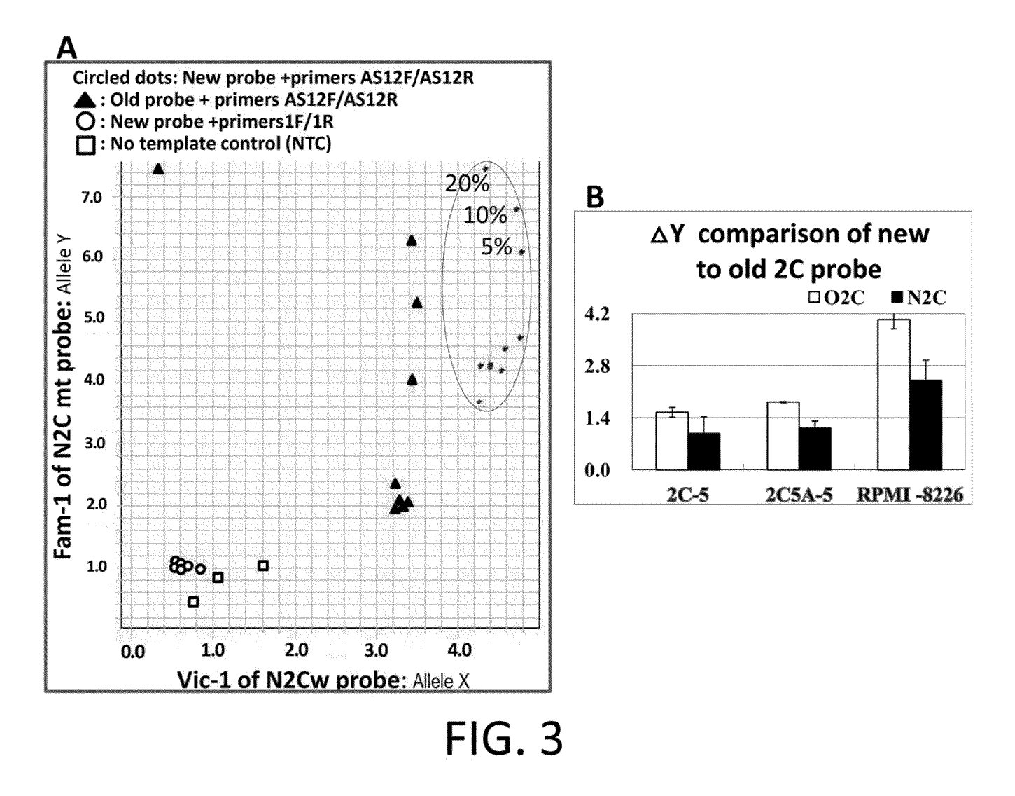

Specimen's Fam intensity (Y) in individual allele reactions was measured against the cutoff point of 1% or 5% mutant DNA (FIGS. 2 and 3). The Vic intensity (X-axis) indicating presence of wild-type allele was expected in all samples unless it was purely a homogeneous mutant. Signals were displayed as either colored demography or numeric data which were exported as excel file. Those with a Y value equal or above the corresponding mutant QC were identified as having the mutation. The bar figures showed average Y values of different experiments.

Although a qualitative assay, allelic discrimination detecting originally 5% QC mutants was improved in detecting the mutant content as low as 1% with an increased Fain intensity difference (ΔY) from wild-type. This process involved changing not only primer sequences to amplify a gene fragment from 80 by to 66 by long but also probe sequences and their pairing (EXAMPLE 4). In the end, probes N1A, N1C, 1T, N2A, 2C, 2T and 5A with primers IF and AS12R were proven to be able to achieve a sensitivity of detecting 1% QC mutants.

The invention proves that both sequences of allelic probes and primers and amplicon's length may affect assay's performance, and furthermore demonstrates the sensitivity and specificity improvement by the sequences combination with different reagents, instruments, and specimen types.

### Example 2

### Comparison of Sensitivity and Specificity to RFLP and Sequencing

In results comparison among three methods, sensitivity is defined as measuring the proportion of actual mutants (regarded as positives) which are correctly identified, and specificity as measuring the proportion of wild-type (regarded as negative) which are correctly identified. Allele result of a method differs from that of two other methods is counted as either false positive or false negative.

$$Sensitivity = \frac{number of true positives}{number of true positives + number of false negatives}$$

$$( the probability of a positive being true positive )$$

$$Specificity = \frac{number of true negatives}{number of true negatives + number of false positives}$$

$$( the probability of a negative being true negative )$$

In primary tests of 21 cancer cell lines with allele discrimination, sequencing and RFLP methods, six were confirmed as K-ras c12 mutants (A549 and H358 at base 1; and SW480, SW 620, RPMI-8226, and H2444 at base 2), eight as c13 mutants (H1355, H1734, and H1755 at base 1; and DLD-1, HCT-8, HCT-116, Lovo, and SW48 at base 2), and seven as K-ras wild-types. RFLP detected one false mutant out of 15 cells (6.7%), which was the result of incomplete digestion (A172) that worsened with the FFPE tissues (n=62) (TABLE 4).

Among the 25 CR tumors identified as K-ras mutants by RFLP, allele discrimination and sequencing verified that 8 were false mutants (8/25=32%), 16 were correct, and 1 had a different mutation (1/17=6% wrong mutant). Due to limited sensitivity (>10% mutant admixture detectable) and poor specificity with CRC specimens (82%, TABLE 4), RFLP was replaced by dHPLC as a third method to verify discrepancies between allele discrimination and DNA sequencing.

There was a 12.4% failure rate of sequencing during handling of the FFPE tissue specimens. Most discrepant results of DNA sequencing displayed noisy demography, some of them with poor PCR production in house prior to send-out. For DNA extracts failed sequencing the first time, double or triple amounts were resubmitted. In our study, sequencing detected more double mutations (TABLE 6) which were proven wrong with a third method and so slightly lowered the sensitivity and specificity (98.8% and 97.5%, respectively, TABLE 5). During sequencing of some older specimens, we observed persistent biased mutations differing with Taq polymerases (Viogene, Qiagene, Invitrogen, and KB HotStart), probably in association with DNA fragmentation as a result of bio-degeneration or poor tissue processing. Newer DNA sequencing technology, such as pyrosequencing and next-generation sequencing (NGS), without PCR step, eliminates the problem.

Outcomes of RFLP assay, dependent on not only PCR manipulations but also digestion of restriction enzymes, are difficult to predict (the poor specificity shown in TABLE 4) and too time consuming. DNA sequencing on FFPE tissues, in spite of a failure rate of 12.4%, kept overall sensitivity (98.8%, TABLE 5) and specificity (97.5%) high enough for clinical application; but its technical limits and vulnerability to errors in processing caused delays and difficulty in troubleshooting. Table 4 shows RFLP results of 62 CRC specimens. Table 5 shows a comparison of allele discrimination and DNA sequencing.

TABLE 4
RFLP

| Sequencing/allele | MT | WT |
|---|---|---|
| discrimination | (mutant) | (wild-type) |
| Confirmed MT | 17 | 0 |
| Confirmed WT | 8 | 37 |

For RFLP, sensitivity = 100% (17/17) and specificity = 82% (37/45).
The RFLP method showed that false MT = 32% (8/25) and wrong MT = 6% (1/17).
Results of sequencing and allele discrimination were used as standards to confirm the RFLP results.

TABLE 5

| | Sequencing MT | Sequencing WT |
|---|---|---|
| Allele discrimination MT | 82 | 1 (5A)* |
| Allele discrimination WT | 3* | 118 |

*dHPLC, the third method, confirmed the genotypes that were
detected by allele discrimination

| | Sequencing | Allele discrimination |
|---|---|---|
| Sensitivity | 82/83 = 98.8% | 83/83 = 100% |
| Specificity | 118/121 = 97.5% | 121/121 = 100% |

### Example 3

### Other Validation of Allele Discrimination

Allele discrimination was verified with a higher sensitivity (100%) and specificity (100%) than sequencing (TABLE 5) by a third method, dHPLC, and the genotyping results were provided in detail in TABLE 6. Among the results that differed from sequencing, allele discrimination proven by dHPLC was correct on one mutant (5A) and three wild-types (sequenced as a wild-type and a 2T and 2A5A and 1C5A double mutations). Although as a reflection of clonal expansion and intratumoral genetic heterogeneity which requires microdissection to verify, double mutations at c12-13 tend to be detected more often with sequencing and SSCP [Bazan et al., 2002; Span M, 1996] than qPCR applications. Overall, K-ras mutations are detected among 83 of 204 CRC specimens (40.7%), with 20.6% of G12D (GAT), 7.4% of 12V (GTT), 7.4% of 13D (GAC), and 5.3% of four other mutations (12C, 12R, 12A, and 12S) combined.

TABLE 6

| | Amino | | | | |
|---|---|---|---|---|---|
| Type | acid | qPCR | Sequencing | n = | % |
| 2G→A | G12D | 44 (−2^{~}) | 44 | 42 | 20.6 |
| 2G→T | G12V | 15 | 16 | 15 | 7.4 |
| 5G→A | G13D | 15 | 18 | 15 | 7.4 |
| 1G→T | G12C | 3 | 3 | 3 | 1.5 |
| 1G→C | G12R | 2 | 3 | 2 | 1.0 |
| 2G→C | G12A | 3 | 3 | 3 | 1.5 |
| 1G→A | G12S | 3 | 4 | 3 | 1.5 |
| Double | | 1A2A*, | 2A5A, 1C5A, | 2 | 1.0 |
| mutations | | 2C2A | 2A5A, 2T5A, | | |
| | | | and two 1A2A | | |
| | | | (one*) | | |
| Sum of mutants | | 85 (−2^{~}) | 91 (−6^{~}) | 83 | 40.7% |
| Wild-type | | 121 (59.3%) | 119 (58.3%) | | |

Two sequenced double mutations, 2A5A and 1C5A (underlined), were detected as wild-types by allele discrimination with dHPLC confirmation. Other discrepancies were three sequenced double mutations—2A5A, 2T5A, and 1A2A—which were detected as single mutations 2A, 2T, and 2A, respectively; and one sequenced single mutation, 2C, which was detected as 2C2A in a mucinous histotype.
*A common double mutation, 1A2A, simultaneously sequenced and detected by the allele discrimination in a metastasized lung.
^{~}Deduction of double mutations.

As the assay's accuracy intra-run precision was analyzed by triplicates of 21 randomly selected patient specimens. And assay's inter-run precision (Chow et al., 2012;) was evaluated with five runs of five CRC specimens including two 2A mutants (P18 and P48), one 2T mutant (P30), and two wild-types (P02 and P04), with the corresponding quality controls (NTC, WT, and 5% mutant plasmids). Each specimen's average Fam (MT) fluorescence values among triplicates or five runs consistently demonstrated small standard deviations (SDs) as evidence of acceptable accuracy and reproducibility. Therefore, clinical validation of the assay including methods comparison and statistical analyses of accuracy and reproducibility of patient's specimens (CLIA guidelines) has been fulfilled.

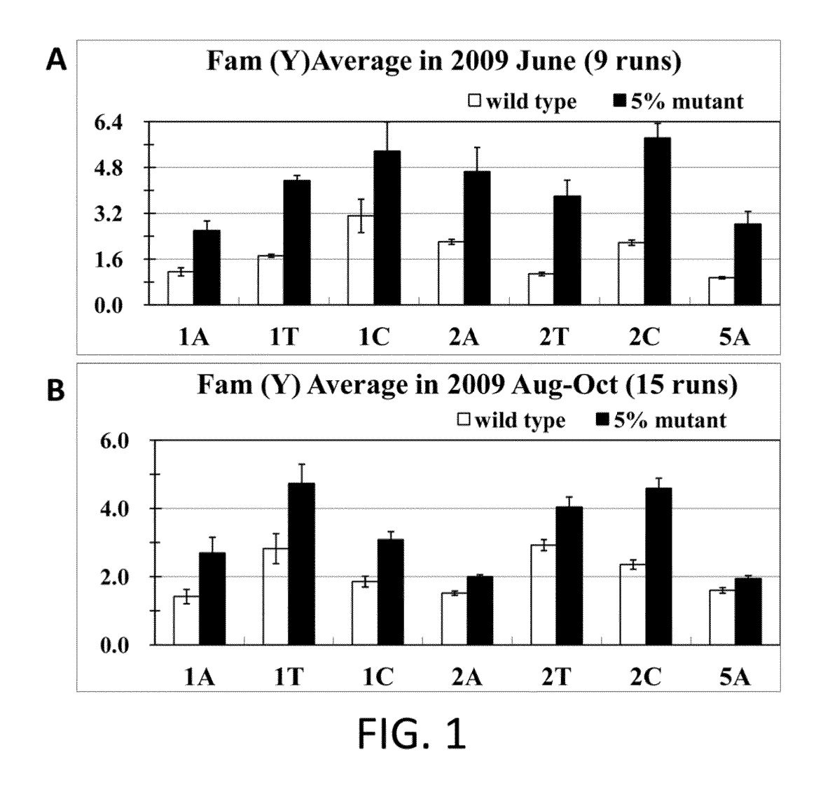

A small group of cholangiacarcinoma specimens (n=40) were also assessed for KRAS mutations and results compared between allele discrimination assay and DNA sequencing. After deduction of 13 specimens that contained less than 5% tumors, the allele discrimination assay detected 2 more mutants and one different mutant than DNA sequencing, i.e., a detection rate of 7% higher. For assay's performance, daily quality control (QC) monitoring is implemented to distinguish the pre-analytical (sample and preparations) errors from the analytical (assay itself) ones, and more importantly, to observe stability of assay's operation (FIG. 1).

### Example 4

### New Allele-Specific Oligonucleotides (ASO) Improved Allele Discrimination

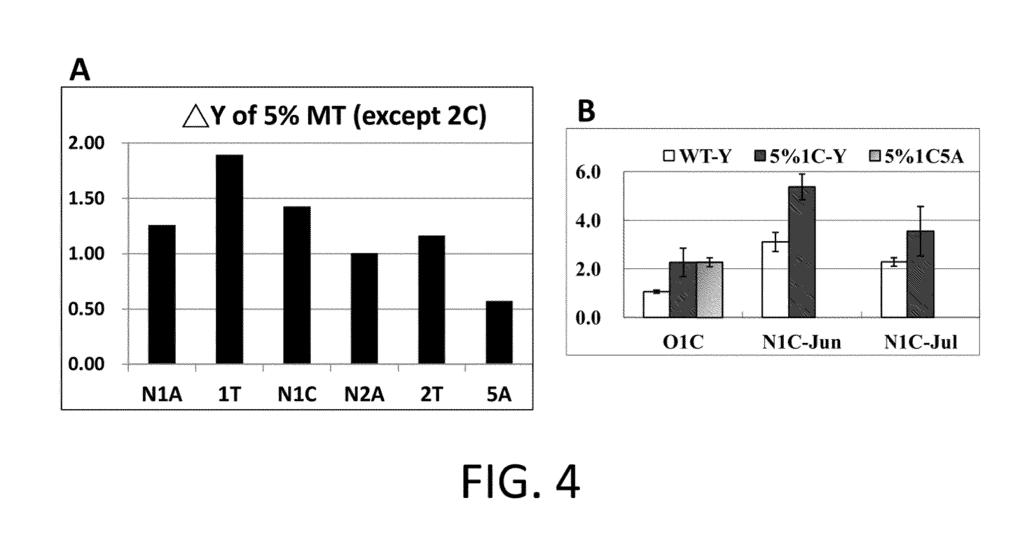

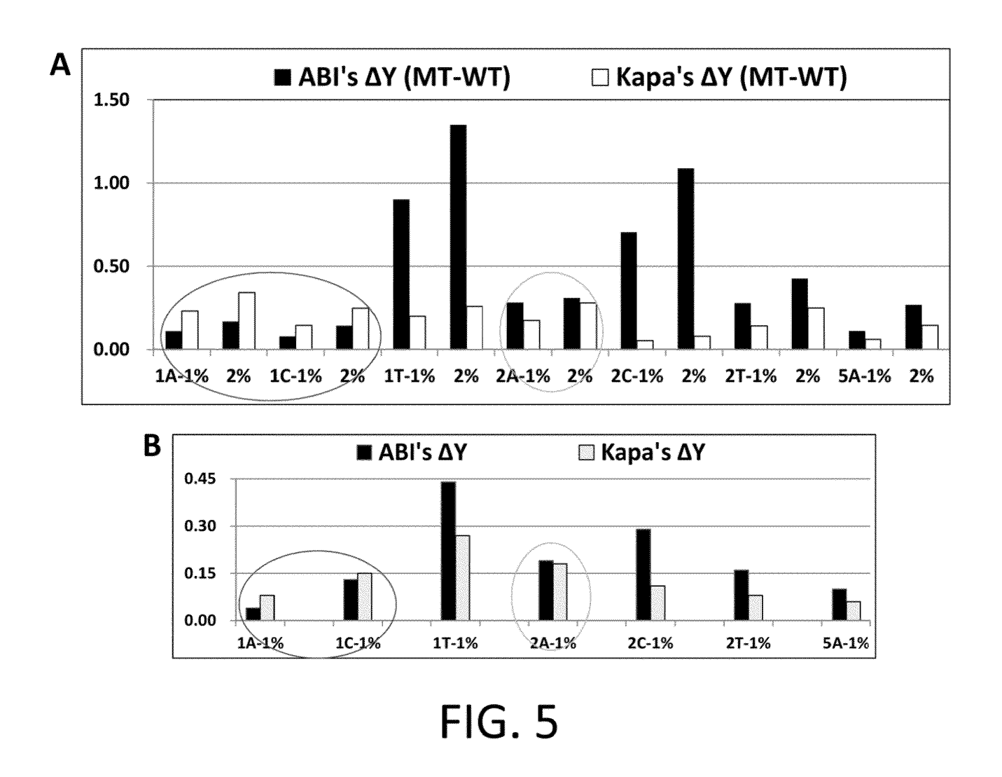

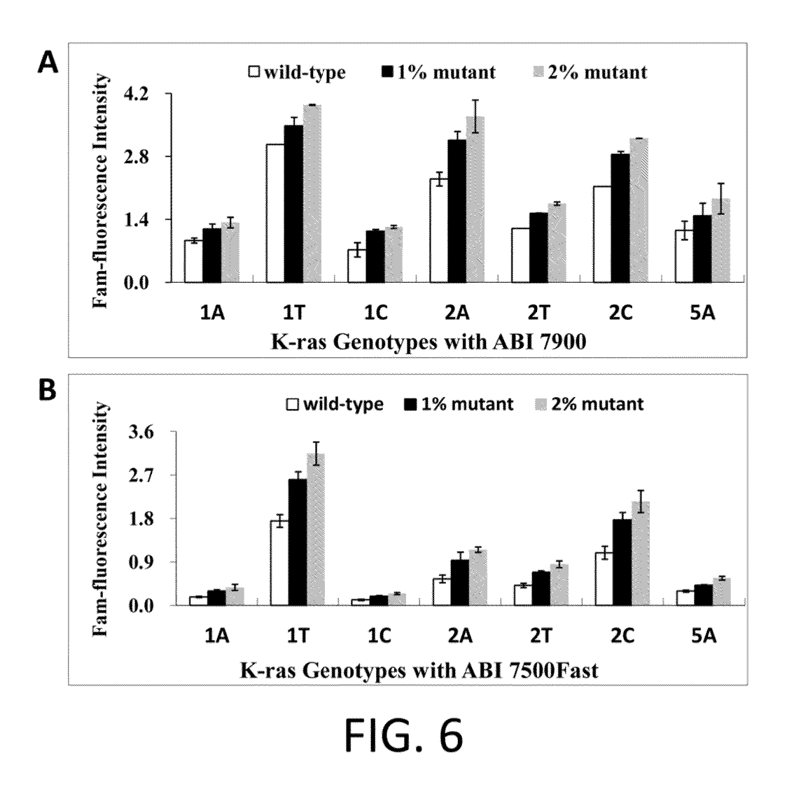

Unlike RFLP and DNA sequencing, which amplify K-ras gene twice, allele discrimination reduces genotyping errors by synchronizing genotype detection with amplification. Its better detection rate and sensitivity during handling of FFPE tissue is conferred by the shorter amplicon (80 by with old primers and 66 by with new primers) with qPCR in contrast to 150 to 250 by with DNA sequencing. New probes (N1A, N1C, N2A, and N2C, TABLE 1) and the primer 1F, paired to AS12R, that gives a 66-bp amplicon instead of 80-bp of primer AS12F, furthermore improved the assay's sensitivity from detecting 5% (FIG. 1) to 1% mutants (FIGS. 5 and 6). In detections of 2A (FIG. 2A) and 2C alleles (FIG. 3A), new N2A and N2C probes were observed to separate the 5 to 100% 2A or 2C mutants (from) better than old ones (A, dots in circles). However, with the Fam (Y) intensity differences (ΔY) between the mutants and wild-type, new 1F primer increased the N2A probe's sensitivity from detecting 5% to 1% 2A mutant (FIG. 2B), but the new, N2C probe's ΔY with the 5% 2C and 2C5A mutant plasmids and the 2C-containing RPMI-8226 cells were not better than the old, 2C probe's (FIG. 3B). Therefore, except 2C, the measureable Y differences (ΔY) of 5% mutants (MT-WT) were displayed in FIG. 4A, and the Fam (Y) intensity of N1C probe was monitored continuously for two months (FIG. 4B).

### Example 5

### Performance Confirmed by Different Taq Polymerases and qPCR Instruments

The new ASO sequences demonstrate comparable results with Kapa Probe Fast qPCR Kit to TaqMan Fast Universal PCR Master Mix (FIG. 5). 1% and 2% mixtures of K-ras mutant plasmids are repetitively used to test the new ASO sequences, and average Fam-fluorescence intensity (Y) of each mutant allele displays responsive increments to mutant percentages on both ABI 7900 and 7500 Fast (FIGS. 6A and 6B).

### Example 6

### Lowered gDNA Requirement and Prospective Applications of the Assay

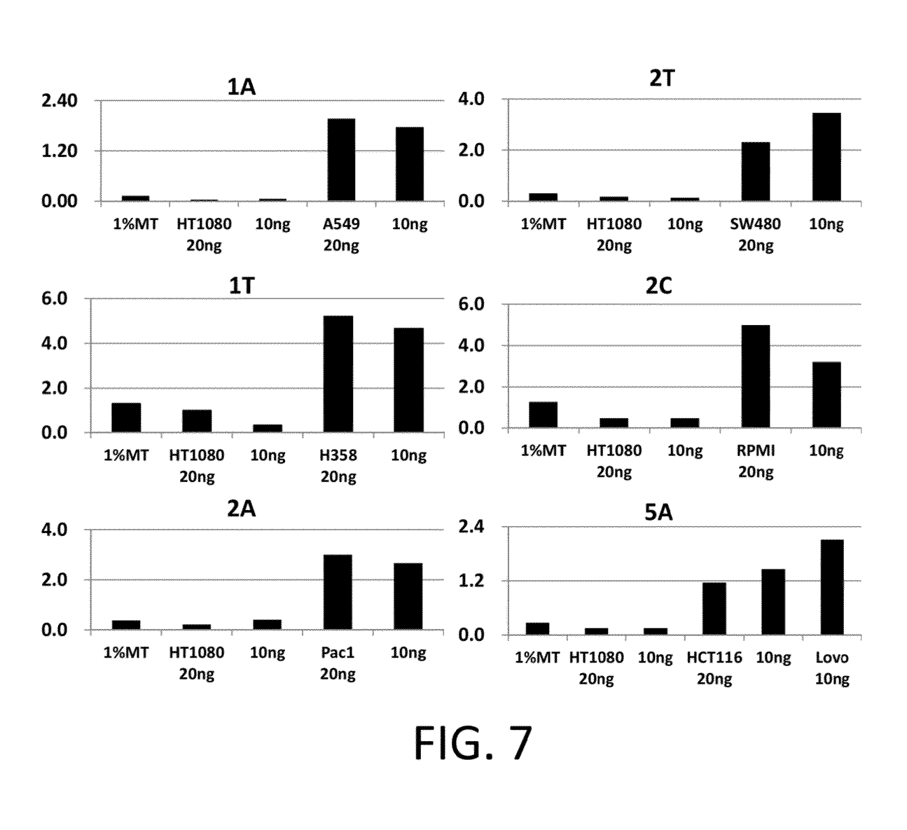

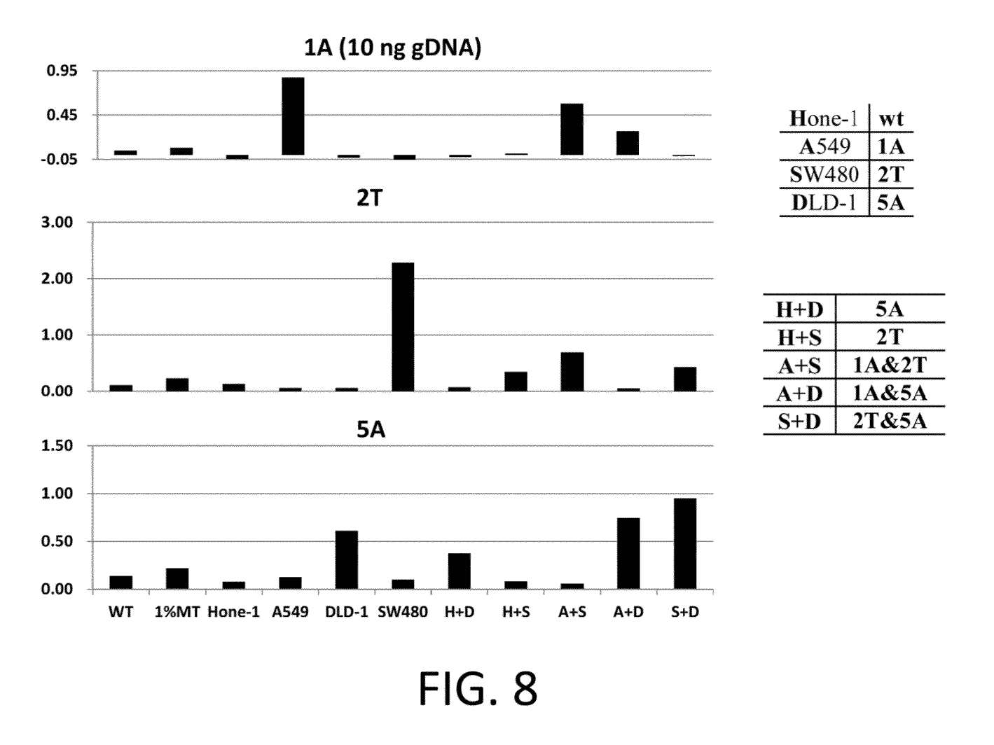

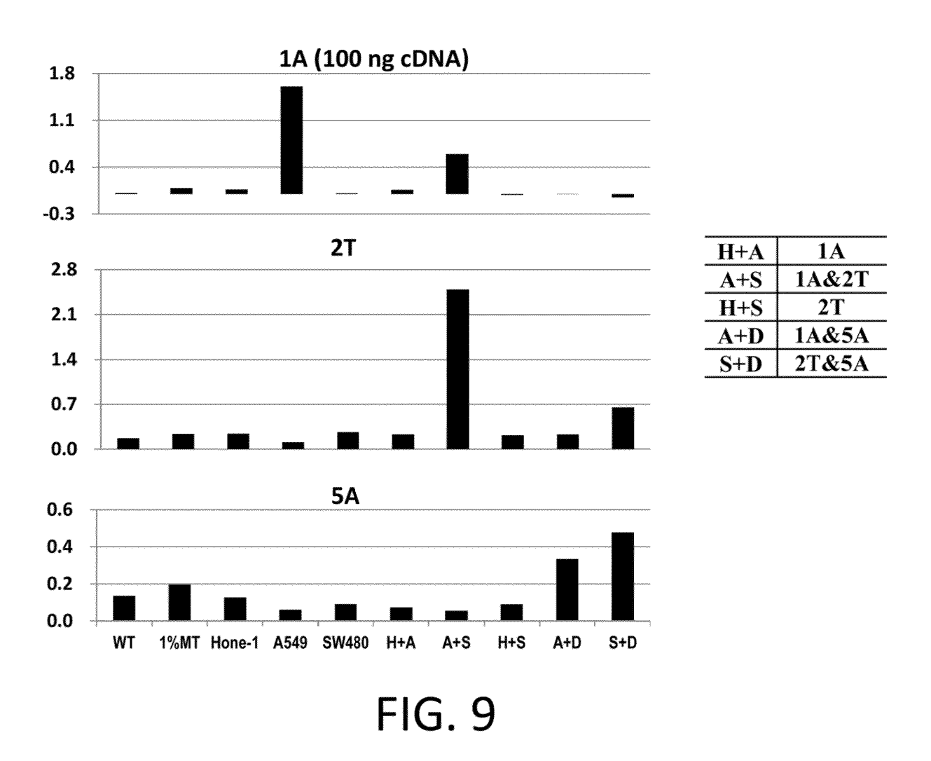

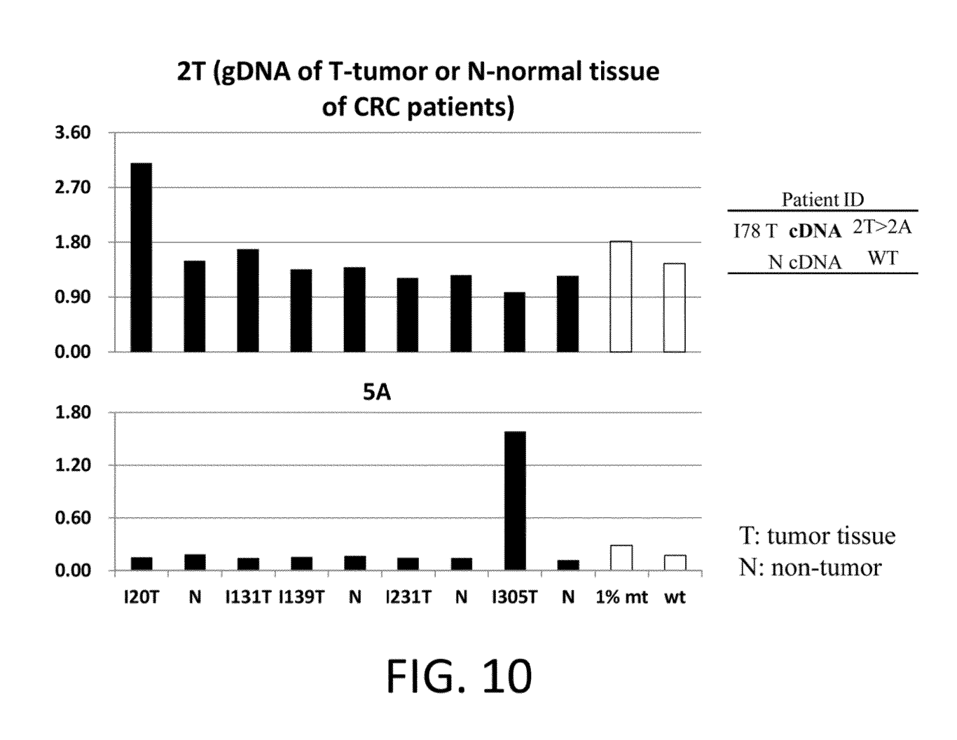

The gDNA amount of cells is lowered from 20 to 10 ng per allele reaction (FIG. 7). The new ASO sequences detected 10 ng gDNA (FIG. 8) and 100 ng cDNA (FIG. 9) of two co-cultured cells with K-ras genotypes corresponding to individual cells. Few serum samples and RNA of frozen tissue from CRC patients were also analyzed. No mutant was detected in 6 serum DNA samples after being stored at −80° C. over a year, in contrast to 2T and 5A mutant found in tumor's gDNA (FIG. 10). The cDNA of reverse transcribed mRNA from a frozen tumor was detected with a 2T and 2A double mutant which lacks gDNA to correlate. In spite of the limited specimens, potential applicability of K-ras assay to circulating DNA, as a non-invasive diagnostic tool, and tissue RNA, may lead to greater clinical impact, for example, faster screening the most current gene alteration in high risk individuals. With simplicity and rapidity our assay is valuable for clinical practice.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A method of diagnosing a KRAS gene mutation at codons 12-13 in a DNA sample, comprising:
detecting one or more than one mutation in the KRAS gene codons 12-13 of the DNA sample by performing an allelic discrimination assay using a mutant probe, a wild-type probe paired with the mutant probe, a forward primer and a reverse primer, the mutant probe detecting a single nucleotide mutation at 1A, 1T, 1C, 2A, 2T, 2C or 5A of the KRAS gene codons 12-13 of the DNA sample, and the primers each having no greater than 25 nucleotides in length for amplifying a region spanning KRAS exon 2 codons 12-13, wherein the mutant and wild-type probes are labeled with different fluorescent dyes.

2. The method of claim 1, wherein the detecting step comprises:
(i) admixing the DNA sample with the paired mutant and wild-type probes, the primers and polymerase chain reaction (PCR) reagents;
(ii) amplifying the region spanning the codons 12-13 of the KRAS exon 2;
(iii) measuring the intensity of the fluorescent dye of the mutant probe in the DNA sample; and
(iv) comparing the intensity of the fluorescent dye of the mutant probe to that of its corresponding quality control (QC) mutant, wherein a comparable or an increased intensity indicates the presence of a mutation.

3. The method of claim 1, wherein the forward and reverse primers comprise the nucleotide sequences of SEQ ID NOs: 14 and 15, respectively.

4. The method of claim 1, wherein the mutant and wild-type probes are a pair selected from the group consisting of:
(i) SEQ ID NO: 1 paired with SEQ ID NO: 2;
(ii) SEQ ID NO: 3 paired with SEQ ID NO: 4;
(iii) SEQ ID NO: 5 paired with SEQ ID NO: 2;
(iv) SEQ ID NO: 7 paired with SEQ ID NO: 8;
(v) SEQ ID NO: 9 paired with SEQ ID NO: 8 or 10;
(vi) SEQ ID NO: 11 paired with SEQ ID NO: 10; and
(vii) SEQ ID NO: 12 paired with SEQ ID NO: 13.

5. The method of claim 4, wherein the forward and reverse primers comprise the nucleotide sequences of SEQ 1D NOs: 14 and 15, respectively.

6. The method of claim 1, wherein the mutant probe is labeled with FAM.

7. The method of claim 6, wherein the wild-type probe is labeled with VIC.

8. The method of claim 1, comprising detecting mutations at 1A, 1T, 1C, 2A, 2T, 2C and 5A of the KRAS gene codons 12-13, wherein the paired probes comprises the following pairs:
(i) SEQ ID NO: 1 paired with SEQ ID NO: 2;
(ii) SEQ ID NO: 3 paired with SEQ ID NO: 4;
(iii) SEQ ID NO: 5 paired with SEQ ID NO: 2;
(iv) SEQ ID NO: 7 paired with SEQ ID NO: 8;
(v) SEQ ID NO: 9 paired with SEQ ID NO: 8 or 10;
(vi) SEQ ID NO: 11 paired with SEQ ID NO: 10; and
(vii) SEQ ID NO: 12 paired with SEQ ID NO: 13.

9. The method of claim 8, wherein the forward and reverse primers comprise the nucleotide sequences of SEQ ID NOs: 14 and 15, respectively.

10. The method of claim 8, wherein the detecting step comprises:
(a) admixing the DNA sample with the paired probes, the primers and polymerase chain reaction (PCR) reagents;
(b) amplifying the region spanning the codons 12-13 of the KRAS exon 2;
(c) measuring the intensities of the fluorescent dye of the respective mutant probes in the DNA sample; and
(d) comparing the intensities of the respective mutant probes to their corresponding quality control (QC) mutants, wherein comparable or increased intensities indicate the presence of mutations.

11. The method of claim I, wherein each probe has no greater than 18 or 16 nucleotides in length.

12. The method of claim 1, wherein the DNA sample comprises a genomic DNA, or cDNA, prepared from a specimen of a tumor biopsy, a paraffin-embedded tumor tissue section (FFPE), a fresh or frozen tumor, or a tumor cell line.

13. The method of claim 12, wherein the tumor biopsy is obtained from a patient with colorectal cancer (CRC), mucinous or metastatic tumor, or cholangiocarcinoma or lung cancer.

14. The method of claim 12, wherein the cDNA is prepared from a frozen or fresh tumor tissue.

15. The method of claim 2, wherein the amplifying step comprises performing a real time PCR.

16. The method of claim 1, wherein the primers are adapted to amplify an amplicon of less than 80 or 70 base pairs.

17. The method of claim 15, wherein the primers are adapted to amplify a DNA fragment comprising the nucleotide sequence of SEQ ID NO: 16.

18. The method of claim 15, wherein the primers are adapted to amplify a DNA fragment consisting of the nucleotide sequence of SEQ ID NO: 16.

19. The method of claim 1, wherein the DNA sample comprises a serum DNA from a CRC patient.

20. The method of claim 12, wherein the amount of gDNA sample is no more than 10 ng/μl.
